(19) United States
(12) Patent Application Publication
(10) Pub. No.: US 2024/0016974 A1
(43) Pub. Date: Jan. 18, 2024

(54) TRANSPARENT DRESSING WITH HYDROGEL LAYER

(71) Applicant: Dukal, LLC, Ronkonkoma, NY (US)

(72) Inventors: Daniel RIVEST, Charlotte, VT (US); Patrick John LAMB, New Port Richey, FL (US); La YANG, Dunedin, FL (US)

(21) Appl. No.: 18/371,682

(22) Filed: Sep. 22, 2023

Related U.S. Application Data
(60) Provisional application No. 63/472,496, filed on Jun. 12, 2023.
(60) Provisional application No. 63/472,146, filed on Jun. 9, 2023.
(60) Provisional application No. 63/172,127, filed on Apr. 8, 2021.
(60) Provisional application No. 63/472,091, filed on Jun. 9, 2023.

(30) Foreign Application Priority Data
Sep. 23, 2022 (CN) 202222528876.6
Jun. 5, 2023 (CN) 2023106537086
Jun. 5, 2023 (CN) 2023214032638

(51) Int. Cl. A61L 26/00; A61F 13/02; A61F 13/00

(52) U.S. Cl. CPC A61L 26/008; A61F 13/0213; A61F 13/00051; A61L 26/0019; A61L 26/0085; A61L 26/0066; A61L 26/0014; A61F 13/0253; A61F 13/0266; A61F 2013/00182; A61L 2300/404

(57) ABSTRACT

A dressing including a top film, an adhesive layer positioned adjacent the top film closer to a user's skin when the bandage is in use, a hydrogel layer including a support layer, below the adhesive layer closer to the user's skin when the bandage is in use, and a lower perforated layer positioned under the hydrogel layer for adhering the bandage to a user's skin. All of the layers may be transparent or at least partially translucent to allow a user to view the user's skin through the bandage. The hydrogel layer provides pressure distribution to reduce stress on the user's skin and reduce the likelihood of pressure ulcers.

### CROSS-REFERENCE TO RELATED APPLICATIONS

The present application claims benefit of and priority to Chinese Utility Model Application No. CN202222528876.6 filed Sep. 23, 2022.

The present application claims benefit of and priority to Chinese Patent Application No. CN2023106537086 filed Jun. 5, 2023.

The present application claims benefit of and priority to Chinese Utility Model Application No. CN2023214032638 filed Jun. 5, 2023.

The present application claims benefit of and priority to U.S. Provisional Patent Application No. 63/472,127 filed Jun. 9, 2023.

The present application claims benefit of and priority to U.S. Provisional Patent Application No. 63/472,091 filed Jun. 9, 2023.

The present application claims benefit of and priority to U.S. Provisional Patent Application No. 63/472,146 filed Jun. 9, 2023.

The present application claims benefit of and priority to U.S. Provisional Patent Application No. 63,472,496 filed Jun. 12, 2023.

### BACKGROUND

### Field of the Disclosure

The present invention relates to a bandage that allows for wound observation while in place on the user's skin. The bandage may include antibacterial material to prevent infection. The bandage may include a hydrogel layer and may be adhered to a user's skin adjacent to a wound via a perforated silicone adhesive layer.

### Related Art

Contemporary medical dressings are typically opaque, so healthcare personnel and patients are unable to directly observe the skin under the bandage. Bandages are commonly used to cover wounds to encourage healing. Bandages may also be used on patients with limited mobility and/or those that are confined to a bed to prevent and treat pressure ulcers which create discomfort and may lead to other serious health problems. Treatment of such ulcers includes covering them with a bandage and close monitoring to avoid infection. The use of opaque bandages, however, does not allow for observation of the wound or ulcer under the bandage, such that frequent removal of the bandage is needed to allow for observation. This frequent removal may create irritation and increases the chance of infection and may affect how quickly the wound heals.

Medical dressings or bandages made of transparent materials have been introduced, however, such bandages have several shortcomings. Due to the material used, adhesive on the dressing sticks to patient's skin after use which may result in discomfort. Further, wound exudate cannot be absorbed by the dressing in a timely manner, thereby affecting the adhesion of the dressing.

The adhesion of the dressings is affected by exudate generated by the wound and may lead to ineffective absorption. Chinese Patent CN201922407013.1, discloses a dressing with improved observing conditions, however, the structure of the bandage results in bubbles forming on its surface which interferes with observation, creates an unfinished appearance and affects long-term storage of these bandages which may lead to waste. Further liquid exudation issues may result from the multilayer structure of the transparent film used in this patent.

Accordingly, it would be beneficial to provide a bandage that avoids these and other problems.

### SUMMARY

It is an object of the present disclosure to provide a bandage including an absorbent hydrogel layer that allows for observation of the user's skin through the bandage.

It is an object of the present disclose to provide a dressing or bandage that absorbs wound exudate while allowing wound observation.

In embodiments, the dressing may include an antibacterial agent which effectively reduces the probability of wound infection.

In embodiments, the dressing includes a protective layer, a silicone gel layer, an absorbent hydrogel layer, an adhesive layer, and a transparent film with one face attached to the face of the adhesive layer opposite the wound area when the bandage is in place.

A medical dressing in accordance with an embodiment of the present disclosure includes: a silicone gel layer including a wound facing surface configured for contact with a user's skin; a hydrogel layer provided above the silicone gel layer and attached to the silicone gel layer, wherein the hydrogel layer includes a support layer supporting hydrogel material; an adhesive layer positioned above the hydrogel layer and attached to the hydrogel layer; and a top film positioned above the adhesive layer, and including a wound facing surface attached to the adhesive layer, wherein the silicone gel layer, hydrogel layer, adhesive layer and top film are transparent to allow observation of a wound through the medical dressing.

In embodiments, the medical dressing includes a protective layer attached to a skin facing surface of the silicone gel layer, opposite the hydrogel layer.

In embodiments, the protective layer is made of polyethylene, polyvinyl chloride, polypropylene, polyethylene glycol terephthalate release film or silicone-coated release paper.

In embodiments, the support layer includes a textured surface in contact with the hydrogel material including one or more recesses configured to receive hydrogel as it expands.

The embodiments, the support layer is a porous material.

In embodiments, the support layer is a thin-film polymer.

In embodiments the support layer is a woven fabric.

In embodiments, the support layer is a nonwoven fabric.

In embodiments, the hydrogel includes an antibacterial agent.

In embodiments, the antibacterial agent is provided as a surface coating on the hydrogel material.

In embodiments, the antibacterial agent is mixed into the hydrogel material.

In embodiments, the antibacterial agent includes one or more of one chlorhexidine gluconate (CHG), polyhexamethylene biguanide hydrochloride (PHMB), benzalkonium chloride (BZK), povidone-iodine, silver nano-sized particles, silver micro-sized particles, silver salt(s) and silver zeolite.

In embodiments, the hydrogel material is acrylamide hydrogel material.

In embodiments, the hydrogel is a hydrophilic, water-swellable, water-insoluble, three-dimensional, cross-linked/polymerized polymer network embedded with a high water content.

In embodiments, the hydrogel layer includes a second support layer positioned opposite the support layer.

In embodiments, the silicone gel layer includes a plurality of openings formed therethrough such that the hydrogel layer is in fluid communication with a wound under the silicone gel layer.

In embodiments, the plurality of openings includes a first group of large openings provided in a center of the silicone gel layer under the hydrogel layer and a second group of small openings formed around an edge of the silicone gel layer.

In embodiments, the adhesive layer is made of a biocompatible material and is used to adhere the antibacterial hydrogel layer and the transparent film.

In embodiments, the adhesive layer is an acrylic acid adhesive, a pressure-sensitive adhesive, a hot-melt adhesive, or an adhesive with the same stickiness.

In embodiments, the top film is a single layer of a polyurethane material.

### BRIEF DESCRIPTION OF THE DRAWINGS

The above and related objects, features and advantages of the present disclosure will be more fully understood by reference to the following, detailed description of the preferred, albeit illustrative, embodiment of the present invention when taken in conjunction with the accompanying figures, wherein:

FIG. 1 is a schematic cross-sectional diagram of a dressing in accordance with an embodiment of the present disclosure.

FIG. 2 is a schematic cross-sectional diagram of the structure of a dressing in accordance with an embodiment of the present disclosure.

FIG. 3 is a schematic cross-sectional diagram of the structure of a dressing in accordance with an embodiment of the present disclosure.

FIG. 4 is a schematic cross-sectional diagram of the structure of a dressing in accordance with an embodiment of the present disclosure.

FIG. 5 is a schematic cross-sectional diagram of the structure of a dressing in accordance with an embodiment of the present disclosure.

FIG. 6 illustrates a cross-sectional diagram of the structure of a dressing in accordance with an embodiment of the present disclosure.

FIG. 7 illustrates a cross-sectional diagram of the structure of a dressing in accordance with an embodiment of the present disclosure.

FIG. 8 illustrates an exploded view of a dressing in accordance with an embodiment of the present disclosure.

FIG. 9 illustrates a perspective view of the dressing of FIG. 8 in accordance with an embodiment of the present disclosure.

FIG. 10 illustrates a perspective view of a dressing in accordance with an embodiment of the present discourse having a circular shape.

### DETAILED DESCRIPTION OF THE EXEMPLARY EMBODIMENTS

A dressing 10 in accordance with an embodiment of the present disclosure may include a protective layer 1, a silicone gel layer 2 with a wound facing surface attached to the protective layer 1, an absorbent hydrogel layer 4 with a wound facing surface attached to the silicone gel layer 2, an adhesive layer 5 attached to the absorbent hydrogel layer 4 and a top film 6 with a wound facing surface thereof attached to the adhesive layer 5. In embodiments, the top film 6 may have a single layer (monolayer) structure to avoid splicing between the layers thereof which could result in leakage of exudate and/or infiltration of bacteria. In embodiments, the protective layer 1 may be removable from the bottom or wound facing surface of the silicone gel layer 2 to allow for application of the dressing 10 to the patient's skin. In embodiments, the protective layer 1 may have a three-layer structure including a lamination film 1a disposed in a middle and an outer film strips 1b, 1c disposed on both sides of the lamination film. In embodiments, the protective layer 1 may include fewer or additional layers.

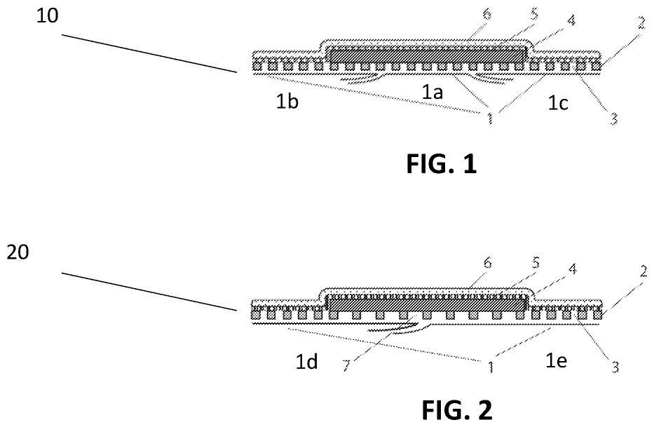

In embodiments, the silicone gel layer 2, hydrogel layer 4, adhesive layer 5, and transparent film 6 are made of transparent materials. In embodiments, the silicone gel layer 2 includes a plurality of openings or air holes 3 extending through the silicone gel layer. In FIG. 1, the air holes 3 are all the same size.

In FIG. 1, the silicone gel layer 2 and the transparent top film 6 are wider and longer than the hydrogel layer 4. The increased size of the silicone gel layer 2 and the transparent film 6 increases the fitting area of the dressing around the wound such that it can be fitted to the wound more easily when in use.

In FIG. 1, the protective layer 1 may be made of a polyethylene material, the top film 6 may be made of a polyurethane material, and the adhesive layer 5 may be made of an acrylic acid adhesive. In embodiments, the protective layer 1 ensures that the silicone gel layer 2 is clean and hygienic prior to use and is easily separated from the silicone gel layer to allow for the silicone gel layer to be applied to a patient's skin.

In embodiments, the protective layer 1 may be removable with the top surface thereof being attached to a bottom, wound facing surface of the silicone gel layer 2. In embodiments, the hydrogel layer 4 may include an antibacterial agent. In embodiments, the silicone gel layer 2, absorbent hydrogel layer 4, adhesive layer 5, and top film 6 are made of a transparent or see-through material to allow observation of the wound, ulcer or skin of the patient through the dressing 10. In embodiments, the antibacterial hydrogel layer 4 may be positioned between the top of the silicone gel layer 2 and the adhesive layer 5. In embodiments, the silicone gel layer 2 includes a plurality of openings or holes 3 that pass completely therethrough. In embodiments, the absorbent hydrogel layer 4 may be in fluid communication with the protective layer 1 through the air holes or openings 3 formed in the silicone gel layer 2 such that the absorbent hydrogel layer is in fluid communication with the wound area through the air holes or openings in the silicone gel layer when the protective layer 1 is removed and the dressing 10 is in place on a wound or ulcer. In embodiments, the absorbent hydrogel layer 4 absorbs wound exudate. In embodiments, the hydrogel layer 4 may have an elastic structure and may deform under pressure, producing elasticity to disperse and relieve the pressure on the wound.

In embodiments, the absorbent hydrogel layer 4 absorbs wound exudate while maintaining a moist environment for the wound. Where antibacterial hydrogel is used, the added antibacterial agent is capable of effectively controlling the microbial load in the area in contact with the wound or ulcer, thereby helping to construct a microenvironment that promotes growth of granulation tissue, promotes healing of the wound surface, and allows the wound to be directly observed in real time. In embodiments, the antibacterial hydrogel layer 4 may be deformed under pressure, producing elasticity to disperse and relieve the pressure on the wound or ulcer, thereby preventing or allowing pressure ulcers to heal.

In embodiments, the adhesive layer 5 may be made of a biocompatible material and may be used to adhere the absorbent hydrogel layer 4 to the transparent top film 6.

In FIG. 1, the silicone gel layer 2 may be made of an organic silicone rubber material.

In embodiments, the top film 1 protects the dressing 10 and the wound surface to limit the chance of infection.

In embodiments, when the hydrogel layer 4 is antibacterial, it may be made of an acrylamide hydrogel material, in which the antibacterial agent is silver micro-sized particles, silver nano-sized particles, silver salt(s) or silver zeolite which may exhibit an antimicrobial efficacy rate that may exceed 99.9%. The specific test data associated with such an antibacterial hydrogel is shown in Table 1 below:

TABLE 1

| | Antimicrobial Efficacy |
|---|---|
| | Rate Exhibited (7 Days |
| Species | of Exposure) |
| Candida albicans | >99.99% |
| Escherichia coli | >99.99% |
| Staphylococcus aureus | >99.99% |
| Enterococcus faecalis | >99.99% |
| Bacillus pneumoniae | >99.99% |
| Pseudomonas aeruginosa | >99.99% |
| Staphylococcus | >99.99% |
| epidermidis | |
| Staphylococcus pyogenes | >99.99% |

In FIG. 1, the dressing may have a liquid absorbency of up to 20 g/100 cm².

In FIG. 1, the dressing may have a water vapor permeability of up to 1,000 g/m²*24 h.

In FIG. 1, the thickness of the antibacterial hydrogel layer is 2.5 mm and the moisture content is 30%.

In FIG. 1, the dressing 10 reduces contact pressure by more than 60%, which is effective in reducing the risk of pressure ulcers. The specific test data is shown in Table 2 below.

TABLE 2

| | | |
|---|---|---|
| | Control (Without | Present Example |
| Sample | Any Dressing) | Is Used |

| | | | | |
|---|---|---|---|---|
| Average force | 261.8 | mmHg | 141.3 | mmHg |
| Largest | 756.3 | mmHg | 286.2 | mmHg |
| Area under force | 0.88 | cm² | 8.8 | cm² |

| | | |
|---|---|---|
| Ratio of reduced contact | — | 62.2% |
| pressure | | |

FIG. 2 illustrates an antibacterial medical dressing 20 in accordance with another embodiment of the present disclosure that allows wound observation. In embodiments, the dressing 20 includes protective layer 1, silicone gel layer 2 with a wound facing surface attached to the protective layer 1, hydrogel layer 4 with a wound facing surface attached to the opposite surface of the silicone gel layer, adhesive layer 5 attached to the opposite surface of the antibacterial hydrogel layer 4, and a transparent top film 6 with a wound facing surface thereof attached to the adhesive layer 5. In FIG. 2, the transparent film 6 has a single (monolayer) structure and the protective layer 1 is removable and uses a two-layer film structure including a left portion 1d and a right portion 1e.

In FIG. 2, the silicone gel layer 2, antibacterial hydrogel layer 4, adhesive layer 5, and transparent film 6 are all made of transparent materials.

In FIG. 2, the air holes formed in the silicone gel layer 2 may be of different sizes with larger air holes 7 and smaller air holes 3. In FIG. 2, the larger air holes 7 are disposed in the center of the silicone gel layer 2 in fluid communication with the hydrogel layer 4 and the smaller air holes 3 are disposed in the application edge area of the silicone gel layer 2.

In FIG. 2, the size of the antibacterial hydrogel layer 4 is smaller than that of the silicone gel layer 2 and the transparent film 6, to provide a larger fitting area around the edge of the dressing 20 such that it fits the wound more easily when in use.

In FIG. 2, the protective layer 1 may be made of a polyethylene material, the transparent top film 6 is made of a polyurethane material, and the adhesive layer 5 is made of acrylic acid.

In FIG. 2, when antibacterial hydrogel is used in the hydrogel layer 4, it is mainly made of an acrylamide hydrogel material, in which the added antibacterial agent is BZK, and may exhibit an antimicrobial efficacy rate that exceeds 90%.

In FIG. 2, the dressing may have a liquid absorbency of up to 10 g/100 cm².

In FIG. 2, the dressing may have a water vapor permeability of up to 1,000 g/m²*24 h.

In FIG. 2, the thickness of the antibacterial hydrogel layer 4 is 2.5 mm and the moisture content is 20%. The dressing 20 reduces contact pressure by more than 50%, which is effective in reducing the risk of pressure ulcers.

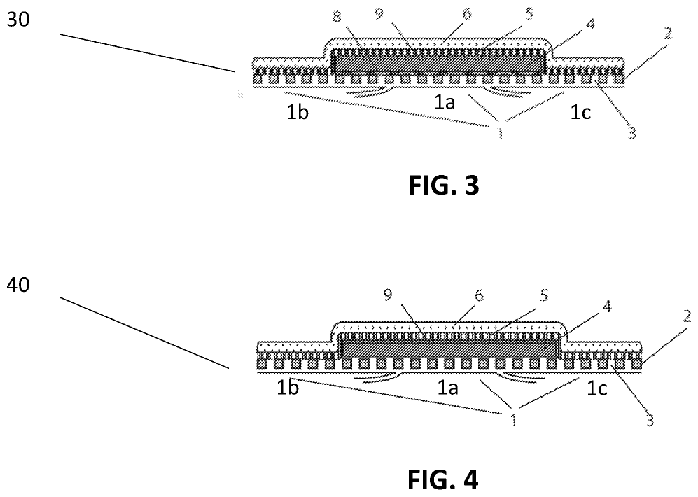

FIG. 3 shows an antibacterial medical dressing 30 in accordance with an embodiment of the present disclosure that also allows wound observation. The dressing 30 includes protective layer 1, silicone gel layer 2 with a surface facing the wound attached to the protective layer 1, hydrogel layer 4 with a surface facing the wound attached to the top surface of the silicone gel layer, adhesive layer 5 attached to the top surface of the hydrogel layer 4 opposite the wound, and top film 6 with a bottom surface thereof attached to the face of the adhesive layer 5 opposite the wound area.

In FIG. 3, a support layer 9 may be disposed on a surface of the hydrogel layer 4 opposite the silicone gel layer 2. In embodiments, the surface of the hydrogel layer 4 in contact with the silicone gel layer 2 may be coated with a high absorption resin coating 8. In FIG. 3, both the support layer 9 and the high absorption resin coating 8 are considered part of the antibacterial hydrogel layer 4.

In FIG. 3, the top film 6 has a monolayer structure. In FIG. 3, the protective layer 1 is removable and has a three-layer structure similar to that discussed above.

In embodiments, the silicone gel layer 2, antibacterial hydrogel layer 4 including the support layer 9, adhesive layer 5, and transparent film 6 are all made of transparent materials. In embodiments, the openings 3 formed in the silicone gel layer 2 may be of the same size. In embodiments, a second support layer (not shown) may be attached to the other side or surface of the hydrogel layer 2.

In FIG. 3, the protective layer 1 may be made of an adhesive-coated release paper material, the transparent top film 6 is made of a polyurethane material, and the adhesive layer 5 is made of hot-melt adhesive.

In FIG. 3, where an antibacterial hydrogel is used in the hydrogel layer 4, it is mainly made of an acrylamide hydrogel material, in which the support layer 9 is a transparent polyurethane film, and in which the antibacterial agent is CHG, and exhibits an antimicrobial efficacy rate that may exceed 99%.

In FIG. 3, the dressing 30 may have a liquid absorbency of up to 40 g/100 cm2.

In FIG. 3, the dressing 30 may have a water vapor permeability of up to 500 g/m2*24 h.

In FIG. 3, the thickness of the hydrogel layer 4 is 2.0 mm and the moisture content is 30%. The dressing 30 of FIG. 3 reduces contact pressure by more than 50%, which is effective in reducing the risk of pressure ulcers.

FIG. 4 shows an antibacterial medical dressing 40 in accordance with an embodiment of the present disclosure that allows wound observation. In FIG. 4, the dressing 40 includes protective layer 1, silica gel layer 2 with the wound facing surface attached to the protective layer 1, antibacterial hydrogel layer 4 with a wound facing surface attached to the surface of the silica gel layer opposite the wound, adhesive layer 5 attached to the surface of the hydrogel layer 4 opposite the wound, and transparent top film 6 with a wound facing surface attached to the adhesive layer 5. In FIG. 4, support layer 9 is disposed on the side of the hydrogel layer 4 opposite the silicone gel layer 2 and the support layer 9 is considered part of the hydrogel layer. In FIG. 4, the transparent top film 6 has a single (monolayer) structure and the protective layer 1 is removable and uses a three-layer structure including a lamination film 1a disposed in the middle and an outer film disposed on both sides 1b, 1c of the lamination film.

In embodiments, the hydrogel layer 4 may be disposed on the support layer 9. In embodiments, the support layer 9 supports the hydrogel during curing and forming of the hydrogel layer 4. In embodiments, the support layer 9 may be coated with one layer of liquid hydrogel during a preparation process, and the liquid hydrogel may be cured and formed under the supporting effect of the support layer 9. In embodiments, the support layer 9 is made of a transparent or translucent material such as a transparent polyurethane film, a non-woven fabric, or a woven or knitted mesh film that is capable of supporting hydrogel curing and allows for observation through the bandage or dressing.

The use of a hydrogel in an absorbent layer of the dressing provides several advantages. For example, hydrogel dressings have the ability to absorb or donate liquid according to the condition of the underlying tissue such that the wound environment remains moist which facilitates cellular migration and proliferation, leading to improved healing times and outcomes. Additionally, hydrogel dressings facilitate autolytic debridement by allowing endogenous enzymes in the wound fluid, secreted by white blood cells, to digest and liquefy necrotic tissue at the wound site. Further, hydrogels have a marked cooling and soothing effect on the skin, which is valuable in burns and painful wounds.

The hydrogel layer 4 may be composed of a hydrophilic, water-swellable, water-insoluble, three-dimensional, cross-linked/polymerized polymer network embedded with a high water content. In embodiments, the hydrogel polymer networks may include synthetic polymers (polyethylene oxide (PO), polyvinyl alcohol (PVA), polyacrylic acid (PAA), polyvinylpyrrolidone (PVP), poly(ethylene) glycol (PEG), polyacrylamide, polypropylene fumarate-co-ethylene glycol (P (PF-coEG), PEO-PEG-PEO, etc.), natural polymers (alginate, chitosan, collagen, gelatin, hyaluronic acid, gellan gum, polyhydroxybutyrate valerate, cellulose, fibrin, pectin, agar, carboxymethylcellulose, to name a few), or a composite of the aforementioned polymers. In embodiments plasticizing agents (Poly(ethylene) glycol, poly(propylene) glycol, silicone oils, etc.) may be included in the hydrogel formulation to improve the flexibility of the material.

In embodiments, the hydrogel polymer network is formed by depositing/pouring the aqueous dispersion of the pre-polymer components onto the support layer 9 and subsequently cross-linking or polymerizing the hydrogel polymer components. There are several approaches by which the hydrogel polymer mixture can be cross-linked or polymerized to form a hydrogel network. The most established and utilized methods of manufacturing hydrogels to be used in wound dressings involves exposing the aqueous hydrogel polymer mixture to ionizing (gamma or e-beam) or UV radiation.

In embodiments, during manufacture of the hydrogel layer 4, the aqueous dispersion of the pre-polymer components often uses a mechanical support layer 9 prior to the cross-linking or polymerization phase of the manufacturing process. The hydrogel is cross-linked, in situ, on the support layer 9 and consequently the hydrogel may form a firm attachment to the support layer. In embodiments, this construction improves the functional integrity and stability of the hydrogel material, especially when incorporating it into a wound dressing. In embodiments, the support layer 9 is typically a thin-film polymer layer (polyurethane (PU), etc.), woven fabrics (nylon mesh, acetate mesh, polyethylene terephthalate (PET) mesh, etc.), or nonwoven fabric (Cellulose, CMC, polypropylene, etc.). In embodiments, the support layer 9 is transparent to allow the observation of the wound or ulcer through the dressing 10, 20, 30, 40, 50, 60, 70 and 80.

A key consideration when selecting a support layer 9 includes identifying a material with sufficient porosity so that the material is breathable (i.e., a MVTR≥500 g/m2 day) to minimize the occurrence of skin maceration during treatment. Another key characteristic is identifying a support layer 9 made of a material that exhibits an appropriate surface tension so that the hydrogel in its pre-crosslinked or pre-polymerized state does not absorb into the support layer. Since hydrogels are generally transparent, a support layer 9 with a material that is also transparent (e.g., a transparent PU film) may offer an additional advantage in that clinicians or patients can monitor the wound site, ulcer or at-risk site on the patient's skin, covered by the dressing without the need to remove it. This reduces the need for unnecessary dressing changes, which can cause wound site trauma and impede the wound healing process. It also reduces the time and cost of clinicians unnecessarily treating an otherwise healing wound or ulcer free site.

In embodiments, the support layer 9 includes a “textured surface” (i.e., including embossed pockets or voids) facing the hydrogel which may improve the overall absorption capacity of the hydrogel material by allowing it to absorb liquid and swell, or expand, into the embossed voids when exposed to exudate. In embodiments, a second support layer may be provided on the opposite side of the hydrogel layer 4.

In FIG. 4, the silicone gel layer 2, antibacterial hydrogel layer 4, including the support layer 9, adhesive layer 5, and transparent film 6 are all made of transparent materials, and the air holes 3 formed in the silicone gel layer 2 are the same size. In embodiments, however, as noted above, the air holes may include larger holes 7 and smaller holes 3 as discussed above.

In FIG. 4, the hydrogel layer 4 is mainly made of an acrylamide hydrogel material and the support layer 9 may be a translucent woven mesh film. In FIG. 4, the antibacterial agent may be PHMB, and the side away from the adhesive layer 4 is coated after the hydrogel is formed to exhibit an antimicrobial efficacy rate that may exceed 99.9%.

In FIG. 4, the dressing 40 may have a liquid absorbency of up to 30 g/100 cm2.

In FIG. 4, the dressing 40 may have a water vapor permeability of up to 1,000 g/m2*24 h.

In FIG. 4, the thickness of the antibacterial hydrogel layer 4 is 5.0 mm and the moisture content is 10%. The dressing 40 reduces contact pressure by more than 50%, which is effective in reducing the risk of pressure ulcers.

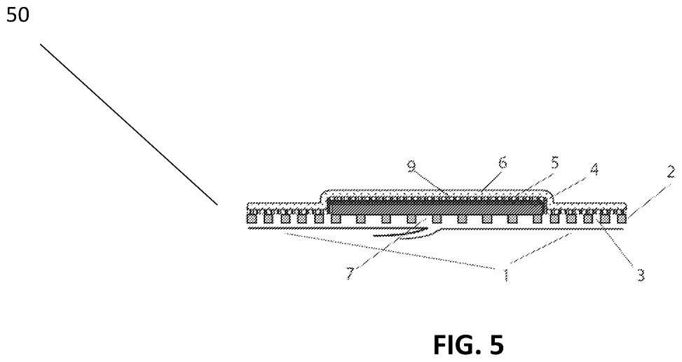

FIG. 5 shows an embodiment of an antibacterial medical dressing 50 in accordance with an embodiment of the present disclosure that allows wound observation. In embodiments, the dressing 50 includes protective layer 1, silicone gel layer 2 with the wound facing surface attached to the protective layer 1, hydrogel layer 4 with a wound facing surface attached to the silica gel layer 2, adhesive layer 5 with a wound facing surface attached to the hydrogel layer 4, and transparent top film 6 with a wound facing surface attached to the face of the adhesive layer 5 opposite the wound area. In FIG. 5, a support layer 9 may be disposed on one side of the antibacterial hydrogel layer 5 away from the silicone gel layer 2 and the support layer 9 is considered part of the antibacterial hydrogel layer. In FIG. 5, the transparent film 6 has a monolayer structure. In FIG. 5, the protective layer 1 is removable and has a two-layer film structure.

In FIG. 5, the silicone gel layer 2, antibacterial hydrogel layer 4, adhesive layer 5, and transparent film 6 and support layer 9 are all made of transparent materials.

In FIG. 5, the air holes formed in the silicone gel layer 2 may include larger air holes 7 and smaller air holes 3 with the larger air holes 7 disposed in the center area in contact with the antibacterial hydrogel layer 4 and the small air holes 3 disposed around the edge of the silicone gel layer 2. The larger holes 7 provided in the area in contact with the wound ensure wound exudate passes through the holes 7 into the hydrogel where it is absorbed and ensure breathability of the hydrogel layer 4. In FIG. 5, the small holes 3 are provided in the edge area of the silicone gel layer 2 to improve adhesion of the dressing 50 and prevent edge warping and adherence difficulties during use.

In FIG. 5, the dressing 50 may have a liquid absorbency of up to 5 g/100 cm2.

In FIG. 5, the dressing 50 may have a water vapor permeability of up to 300 g/m2*24 h.

In FIG. 5, the thickness of the antibacterial hydrogel layer 2 is 0.8 mm and the moisture content is 90%. The dressing 50 reduces contact pressure by more than 40%, which is effective in reducing the risk of pressure ulcers.

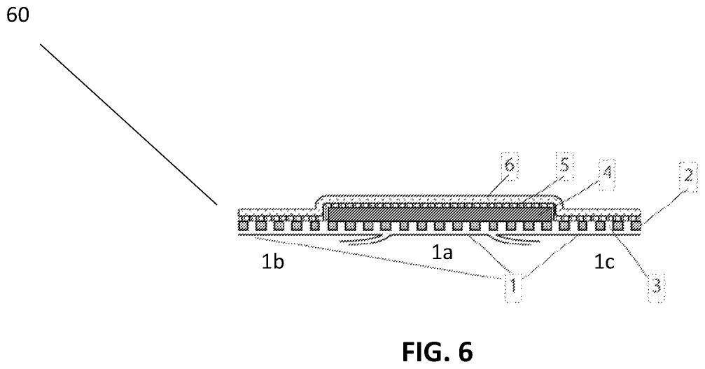

In embodiments, a medical dressing 60 (see FIG. 6) in accordance with an embodiment of the present disclosure allows for observing conditions of a wound under the dressing without removing the dressing from the patient. In FIG. 6, the dressing 60 includes protective film 1, silicone gel layer 2 with a wound facing surface attached to the protective film 1, a hydrogel layer 4 with a wound facing surface attached to the silicone gel layer, acrylic adhesive layer 5 with a wound facing surface attached to the hydrogel layer 4 and another surface attached to transparent top film 6, where the transparent top film 6 is of a single-layer structure. In embodiments, the protective film 1 is tearable for removal and includes a left film 1b arranged on the left side, a right film 1c arranged on the right side and a bonding film 1a arranged in the middle of the left film and right film.

In embodiments, the silicone gel layer 2, the hydrogel layer 4, the acrylic adhesive layer 5 and the transparent film 6 are all made of a transparent material. In the embodiments, the silicone gel layer 2 is provided with air holes 3 formed entirely therethrough while the acrylic adhesive layer 5 is not provided with holes.

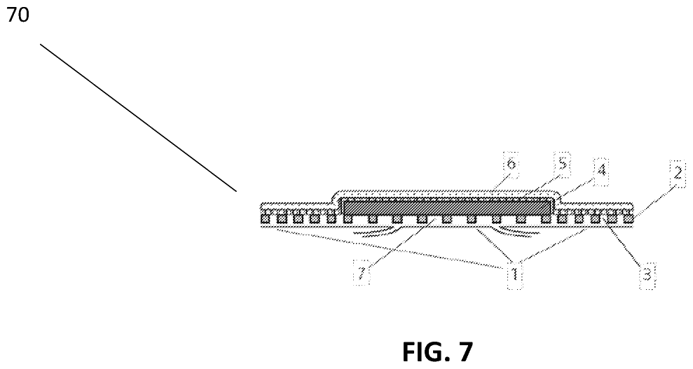

In FIG. 7, the dressing 70 is similar to the dressing 60 except that the silicone gel layer 2 includes larger air holes 7 and smaller air holes 3, where the larger air holes 7 are formed in the silicone gel layer 2 in the center area under the hydrogel layer 4 and the smaller air holes 3 are formed in an edge area of the silicone gel layer.

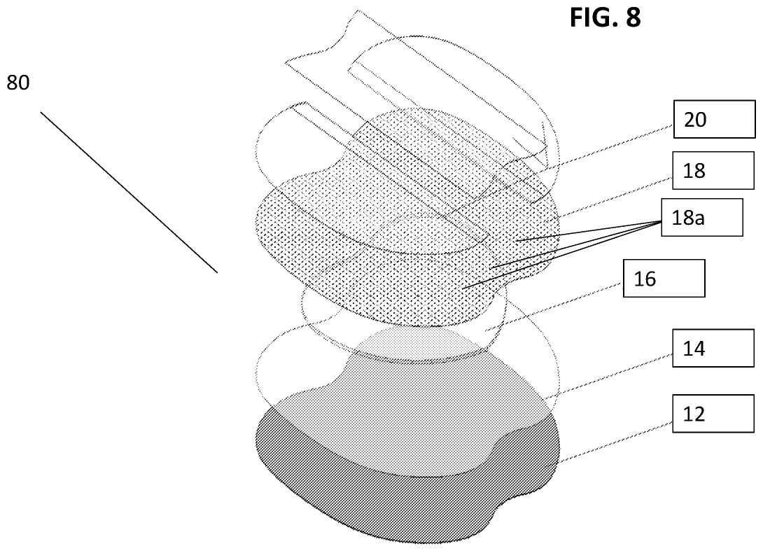

A dressing 80 in accordance with another embodiment of the present disclosure is illustrated in FIG. 8. In embodiments, the dressing 80 may include a top film 12 made of a transparent polyurethane, however, the film 12, like the film 1 discussed above, is not limited to this material and may be made of any suitable material. In embodiments, the top film 12, like the film 1 discussed above, may be transparent or substantially transparent such that the skin under the bandage 80 may be visible. In embodiments, the top film 12 may be translucent to allow at least partial vision of the skin under the bandage 80.

In embodiments, an adhesive layer 14 may be provided adjacent to the top film 12, closer to the patient's skin when the bandage 10 is in place on the user's body. In embodiments, the adhesive layer 14 secures the top film 12 to the bandage 80 and prevents buckling of the bandage similar to the adhesive layer 5 discussed above. In embodiments, the adhesive layer 14 may be a layer of adhesive provided as a coating on a body facing surface of the film 12. In embodiments, the adhesive layer 14 may be a separate layer of material. In embodiments, the adhesive layer 14 may be or use an acrylic adhesive, however, any suitable adhesive may be used including those used for adhesive layer 5. In embodiments, the adhesive layer 14 may be transparent to allow a wound covered by the bandage 80 to be viewed through the bandage. In embodiments, the adhesive layer 14 may be transparent or translucent to allow observation of the skin under the bandage 10.

In embodiments, the adhesive layer 14 connects the top film 12 to an absorbent layer 16. In embodiments, the absorbent layer 16 may be or include a hydrogel, similar to the hydrogel layer 4 discussed above and absorbs moisture from the ulcer or wound to which the bandage 80 is applied. In embodiments, the absorbent layer 16 has a thickness of between 2 mm and 3 mm. In embodiments, the absorbent layer 16, which may be the hydrogel layer 4 discussed above, is sufficiently absorbent to absorb substantial fluid from the ulcer or wound without requiring replacement, since frequent removal of the bandage 80 increases the risk of infection. In embodiments, the absorbent layer 16 may be sufficiently absorbent to remain in place on the patient for extended periods of time.

In embodiments, the hydrogel material of the absorbent layer 16 provides for pressure distribution which reduces the likelihood that a pressure ulcer will form. In embodiments, the absorbent layer 16, including the hydrogel material, may be transparent to allow a wound covered by the bandage 80 to be viewed through the bandage. In embodiments, the absorbent layer 16 may be translucent to allow at least partial vision of the skin under the bandage 80.

In embodiments, a lower layer 18 is provided below the absorbent layer 16 when the bandage 80 is in place and contacts the user's skin. In embodiments, the lower layer 18 is the silicone gel layer 2 discussed above. In embodiments, the lower layer 18 may be perforated to allow fluid from the wound to pass through it and into the hydrogel of absorbent layer 16. In embodiments, the lower layer 18 may be or may include silicone gel. In embodiments, the adhesive lower layer 18 may be a polyurethane film with a silicone-based adhesive provided thereon to secure the bandage 80 to the user's skin. In embodiments, an acrylic based adhesive may be provided on the upper surface of the lower layer 18 to secure it to the absorbent layer 16 however, any other adhesive may be used. In embodiments, the adhesive or material in contact with the user's skin is silicone based. In embodiments, the silicone adhesive on the lower surface of the adhesive lower layer 18 adheres to the user's skin around the wound while avoiding a strong bond with the wound. In embodiments, other materials may be used to make the layer 18, provided that the adhesive that faces the user's skin is silicone based.

In embodiments, the lower layer 18 includes perforations 18a that extend therethrough to allow fluid to pass into the absorbent layer 16 from the user's skin. In embodiments, the perforations 18a may be similar to the openings 3, 7 discussed above. In embodiments, the lower layer 18 provides for a secure connection to the user's skin and allows for relatively easy removal, without damaging the wound or ulcer.

In embodiments, the lower layer 18, may be transparent to allow a wound covered by the bandage 10 to be viewed through the dressing 80. In embodiments, the lower layer 18 may be translucent to allow at least partial vision of the skin under the bandage 10.

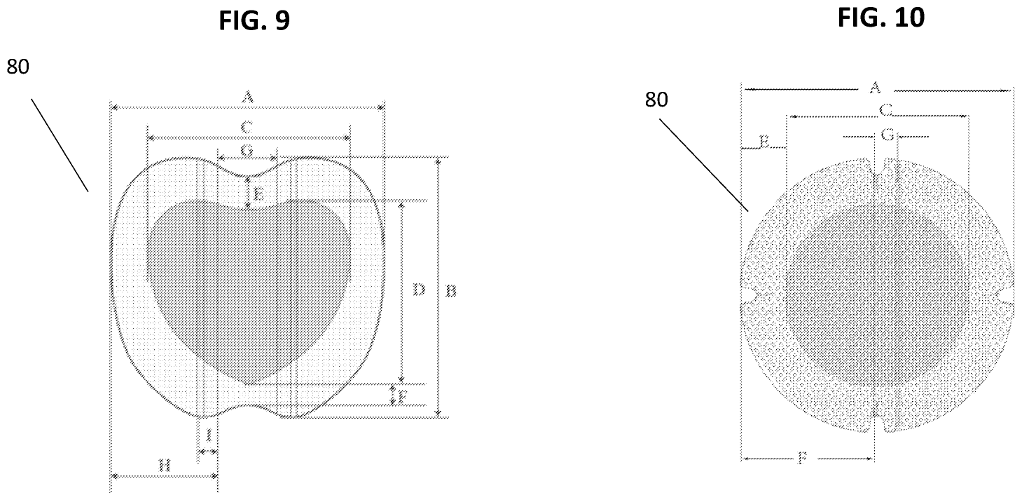

In embodiments, the bandage 80 may be made in a variety of shapes. FIG. 9 illustrates a heart-shaped dressing and exemplary dimensions as follows.

| | | | | | | | | | |
|---|---|---|---|---|---|---|---|---|---|
| Item | Dressing | Dressing | Pad | Pad | Pad to | Pad to | Middle | Edge to | PE liner |
| | Width | Length | Width | Length | edge | edge | liner | fold- | Fold |
| | (A) | (B) | (C) | (D) | (E) | (F) | Length | center(H) | Length |
| | | | | | | | (G) | | (I) |

FIG. 10 illustrates an embodiment of the bandage 80 having a substantially circular shape. The dimensions illustrated FIGS. 9-10 are in mm and are exemplary such that they may be varied to provide smaller or larger bandages.

In embodiments, one or more protective liners 20 may cover the silicone adhesive provided on the patient contacting lower layer 18 until the bandage 80 is applied to the user's skin like the protective film 1 discussed above. In embodiments, the liners may be made of polyethylene, however, any other suitable material may be used provided that the liners are removable from the lower layer 18 without damaging the adhesive.

In embodiments, a packaging element, or elements, (not shown) may be provided to store the bandage 80 before use.

The dressing 80 provides advantages in that the use of transparent, or at least substantially translucent materials for all layers allows the wound, or the user's skin under the bandage to be visible without removing the bandage. This allows for monitoring of wound healing without removal of the bandage. Similarly, where the bandage 80 is being used to prevent formation of pressure ulcers, the visibility through the bandage allows monitoring of the patient's skin without removal of the bandage to ensure that ulcers are not forming. Further, the use of the hydrogel material in the absorption layer 16 provides for pressure distribution to minimize the pressure conveyed to the user's skin and reduce the likelihood of pressure ulcer formation.

In embodiments, the dressing 10, 20, 30, 40, 50, 60, 70 and 80 may be provided in different shapes and sizes depending on the location of the end-use wound or ulcer. In embodiments, the dressing may be heart-shaped (see FIG. 9), circular (see FIG. 10), elliptical, triangular or rectangular, to name a few, to accommodate different wound or ulcer locations or shapes. For example, when the dressing is used for wounds at the tailbone, a heart shape may be used.

In embodiments, the antibacterial agent may be one or several of chlorhexidine gluconate (CHG), polyhexamethylene biguanide hydrochloride (PHMB), benzalkonium chloride (BZK), povidone-iodine, silver nano-sized particles, silver micro-sized particles, silver salt(s) and silver zeolite to name a few.

In embodiments, the antibacterial agent may be added by surface coating after hydrogel forming or added directly during hydrogel forming. In embodiments, the antibacterial hydrogel is absorbent. In embodiments, the antibacterial hydrogel may include an absorbent or superabsorbent polymer.

In embodiments, the thickness of the hydrogel layer may be 0.8-5 mm and the moisture content of the antibacterial hydrogel layer is 10-90%. The thickness and moisture content allow pressure on the wound to be effectively dispersed and relieved to relieve pressure on the user's skin.

In embodiments, the protective layer 1 may be made of polyethylene, polyvinyl chloride, polypropylene, polyethylene glycol terephthalate release film, silicone-coated release paper, and the like.

In embodiments, the adhesive layer 5 may be an acrylic adhesive, pressure-sensitive adhesive, hot-melt adhesive, or other adhesives with the same stickiness.

In embodiments, the acrylic adhesive layer 5 may not be provided with air holes, and the transparent film 6 is of a single-layer structure to eliminate bubbles on the surface of the dressing which affects appearance and long-term storage.

In embodiments, providing improved adhesion around the edge of the silicone gel layer 2 may prevent edge warping and adherence difficulties during use, thereby improving the patient's comfort. In embodiments, the larger air holes may be 0.5-5.0 mm and the small air holes may be 0.1-2.0 mm.

In embodiments, the silicone gel layer 2 and hydrogel used in the absorbent hydrogel layer 4 provide a wet environment that protects a surface of the wound and provides pain relief. In embodiments, as noted above, the use of hydrogel also reduces pressure on the wound which helps to treat and prevent pressure ulcers.

Providing the acrylic adhesive layer 5 without openings or air holes and using a single (monolayer) transparent top film 6 improves appearance and resolves long term storage issues and reduces the risk of liquid exudate leakage and bacterial entry caused by splicing of a multiple layer transparent film.

In embodiments, the dressing 10, 20, 30, 40, 50, 60, 70 and 80 of the present disclosure provides several benefits: (1) the body structure is made of a transparent or see-through material, such that the healthcare personnel and patients are able to observe the wound, ulcer, or user's skin without removing the dressing; (2) by adding antibacterial agents to the hydrogel layer, the probability of wound infection is effectively reduced; (3) the multilayer structure, absorbency of the hydrogel (with or without the coating of the hydrogel layer with a high absorption resin layer) greatly improves wound exudate absorbency of the dressing; (4) contact pressure on the wound, ulcer or user's skin may be dispersed and reduced, which effectively reduces the risk of pressure ulcers; and (5) there is no residual adhesive, which prevents adhesion between wound tissue and the dressing.

Although the present invention has been described in relation to particular embodiments thereof, many other variations and modifications and other uses will become apparent to those skilled in the art. It is preferred, therefore, that the present invention be limited not by the specific disclosure herein.

What is claimed is:

1. A medical dressing comprising:
a silicone gel layer including a wound facing surface configured for contact with a user's skin;
a hydrogel layer provided above the silicone gel layer and attached to the silicone gel layer, wherein the hydrogel layer includes a support layer supporting hydrogel material;
an adhesive layer positioned above the hydrogel layer and attached to the hydrogel layer; and
a top film positioned above the adhesive layer, and including a wound facing surface attached to the adhesive layer,
wherein the silicone gel layer, hydrogel layer, adhesive layer and top film are transparent to allow observation of a wound through the medical dressing.

2. The medical dressing of claim 1, further comprising a protective layer attached to a skin facing surface of the silicone gel layer, opposite the hydrogel layer.

3. The medical dressing of claim 2, wherein the protective layer is made of polyethylene, polyvinyl chloride, polypropylene, polyethylene glycol terephthalate release film or silicone-coated release paper.

4. The medical dressing of claim 1, wherein the support layer includes a textured surface in contact with the hydrogel material including one or more recesses configured to receive hydrogel as it expands.

5. The medical dressing of claim 1, wherein the support layer is a porous material.

6. The medical dressing of claim 1, wherein the support layer is a thin-film polymer.

7. The medical dressing of claim 1, wherein the support layer is a woven fabric.

8. The medical dressing of claim 1, wherein the support layer is a nonwoven fabric.

9. The medical dressing of claim 1, wherein the hydrogel includes an antibacterial agent.

10. The medical dressing of claim 9, wherein the antibacterial agent is provided as a surface coating on the hydrogel material.

11. The medical dressing of claim 9, wherein the antibacterial agent is mixed into the hydrogel material.

12. The medical dressing of claim 1, wherein the antibacterial agent includes one or more of chlorhexidine gluconate (CHG), polyhexamethylene biguanide hydrochloride (PHMB), benzalkonium chloride (BZK), povidone-iodine, silver nano-sized particles, silver micro-sized particles, silver salt(s) and silver zeolite.

13. The medical dressing of claim 1, wherein the hydrogel material is acrylamide hydrogel material.

14. The medical dressing of claim 1, wherein the hydrogel is a hydrophilic, water-swellable, water-insoluble, three-dimensional, cross-linked/polymerized polymer network embedded with a high water content.

15. The medical dressing of claim 1, wherein the hydrogel layer includes a second support layer positioned opposite the support layer.

16. The medical dressing of claim 1, wherein the silicone gel layer includes a plurality of openings formed therethrough such that the hydrogel layer is in fluid communication with a wound under the silicone gel layer.

17. The medical dressing of claim 16, wherein the plurality of openings includes a first group of large openings provided in a center of the silicone gel layer under the hydrogel layer and a second group of small openings formed around an edge of the silicone gel layer.

18. The medical dressing of claim 1, wherein the adhesive layer is made of a biocompatible material and is used to adhere the antibacterial hydrogel layer and the transparent film.

19. The medical dressing of claim 1, wherein the adhesive layer is an acrylic acid adhesive, a pressure-sensitive adhesive, a hot-melt adhesive, or an adhesive with the same stickiness.

20. The medical dressing of claim 1, wherein the top film is a single layer of a polyurethane material.
